(19) United States
(12) Patent Application Publication
(10) Pub. No.: US 2023/0330995 A1
(43) Pub. Date: Oct. 19, 2023

(54) LIQUID DISCHARGE APPARATUS, LIQUID DISCHARGE CONTROL METHOD, AND FILLING METHOD

(71) Applicant: SONY GROUP CORPORATION, TOKYO (JP)

(72) Inventor: SHOGO HARADA, TOKYO (JP)

(21) Appl. No.: 18/041,915

(22) PCT Filed: Aug. 12, 2021
(86) PCT No.: PCT/JP2021/029735

§ 371 Date: Feb. 16, 2023

(30) Foreign Application Priority Data
Aug. 27, 2020 (JP) 2020-143289

(51) Int. Cl. B41J 2/175

(52) U.S. Cl. CPC B41J 2/17506; B41J 2/17596

(57) ABSTRACT

[Object] To provide a liquid discharge apparatus, a liquid discharge control method, and a filling method including a pump using a tube, which realize high-accuracy liquid discharge.

[Solving Means] A liquid discharge apparatus includes a liquid transfer pump, a first flow channel, a second flow channel, and a flow channel switching unit. The liquid transfer pump includes a flexible tube that allows a liquid to circulate therethrough, the liquid being supplied from a liquid storage unit that stores the liquid, a pressing unit that presses the flexible tube from an outer circumferential surface side and feeds the liquid from one end to another end of the flexible tube, and a drive unit that rotationally drives the pressing unit. A first flow channel and a second flow channel that are different from each other are flow channels to which a liquid transferred from the liquid transfer pump flows out. The flow channel switching unit is controlled to use, as the first flow channel, a flow channel to which a liquid for discharge that is transferred from the liquid transfer pump flows out and use, as the second flow channel, a flow channel to which a liquid transferred from the liquid transfer pump flows out until the drive unit moves to a position after the liquid for discharge is transferred, the position being a position where the drive unit is located when the liquid for discharge starts to flow out.

### TECHNICAL FIELD

The present technology relates to a liquid discharge apparatus, a liquid discharge control method, and a filling method.

### BACKGROUND ART

A volumetric pump capable of discharging with high accuracy, e.g., a plunger pump repeats volume measurement and discharge by a syringe portion or plunger portion fabricated at a high working accuracy in order to ensure its discharge accuracy. However, such a worked product is expensive and requires a long production time, so it is unrealistic to dispose of all wetted portions. Therefore, in a case of discharging different kinds of liquid by the use of the same pump, a washing process and a sterilization process are required for the changeover, and there are problems related to workability, safety, and contamination possibility, and so on.

Moreover, one of pumps with disposable wetted portions is a peristaltic pump with a tube (e.g., see Patent Literature 1). The peristaltic pump employs a system in which a flexible liquid-feeding tube is squeezed by the use of a component such as a roller and a liquid inside the tube is pushed out. The peristaltic pump is inferior in liquid-feeding accuracy and dispensing accuracy to the above-mentioned volumetric pump, and is not suitable for applications where high repeatability and reproducibility are desired.

Furthermore, it is desirable to dispose of wetted portions in a variety of fields such as medical, food, and manufacturing industries.

### CITATION LIST

### Patent Literature

Patent Literature 1: Japanese Patent Application Laid-open No. 2015-181550

### DISCLOSURE OF INVENTION

### Technical Problem

As described above, a liquid discharge apparatus including the pump with the tube has a problem in that it is difficult to discharge a liquid with high accuracy.

In view of the above-mentioned circumstances, it is an objective of the present technology to provide a liquid discharge apparatus including a pump with a tube, a liquid discharge control method, and a filling method that realize high-accuracy liquid discharge.

### Solution to Problem

In order to accomplish the above-mentioned objective, a liquid discharge apparatus according to an embodiment of the present technology includes a liquid transfer pump, a first flow channel, a second flow channel, and a flow channel switching unit.

The liquid transfer pump includes a flexible tube that allows a liquid to circulate therethrough, the liquid being supplied from a liquid storage unit that stores the liquid, a pressing unit that presses the flexible tube from an outer circumferential surface side and feeds the liquid from one end to another end of the flexible tube, and a drive unit that rotationally drives the pressing unit.

The first flow channel is a flow channel to which a liquid transferred from the liquid transfer pump flows out.

The second flow channel is a flow channel to which a liquid transferred from the liquid transfer pump flows out, the second flow channel being different from the first flow channel.

The flow channel switching unit has a flow channel to which a liquid transferred from the liquid transfer pump flows out as the first flow channel or the second flow channel, the flow channel switching unit being controlled to use, as the first flow channel, a flow channel to which a liquid for discharge that is transferred from the liquid transfer pump flows out and use, as the second flow channel, a flow channel to which a liquid transferred from the liquid transfer pump flows out until the drive unit moves to a position after the liquid for discharge is transferred, the position being a position where the drive unit is located when the liquid for discharge starts to flow out.

With such a configuration, the discharge is performed so that the position of the flexible tube that is pressed by the pressing unit is constantly the same position every time. The discharge rate at every time can be thus controlled with high accuracy.

The drive unit of the liquid transfer pump may be driven on the basis of a pulse signal, and
assuming that a number of pulses of the pulse signal for discharge, which is for transferring the liquid for discharge, is defined as m (m>0) and a number of pulses of the pulse signal, which is necessary for the drive unit to rotate once, is defined as n (n>0),
in a case where n<m,
the flow channel switching unit may be controlled to use, as the first flow channel, a flow channel to which a liquid transferred from the liquid transfer pump driven on the basis of the pulse signal for discharge flows out and use, as the second flow channel, a flow channel to which a liquid transferred from the liquid transfer pump driven on the basis of the pulse signal with a number of pulses b satisfying m=an+b (a denotes one or more integers, b>0, n>b) flows out after the liquid for discharge is transferred, and
in a case where n>m,
the flow channel switching unit may be controlled to use, as the first flow channel, a flow channel to which a liquid transferred from the liquid transfer pump driven on the basis of the pulse signal for discharge flows out and use, as the second flow channel, a flow channel to which a liquid transferred from the liquid transfer pump driven on the basis of a pulse signal with a number of pulses c satisfying c=n−m flows out after the liquid for discharge is transferred.

The liquid discharge apparatus may further include a control unit that controls the flow channel switching unit.

The liquid discharge apparatus may further include a detection unit that detects a change in position of the drive unit, in which the control unit may control the flow channel switching unit on the basis of a detection result of the detection unit.

The control unit may further control an operation of the drive unit, and
the control unit may
cause, in a case where n<m, the drive unit to operate on the basis of the pulse signal for discharge and then operate on the basis of the pulse signal with the number of pulses b, and
cause, in a case where n>m, the drive unit to operate on the basis of the pulse signal for discharge and then operate on the basis of the pulse signal with the number of pulses c.

The second flow channel may cause a liquid transferred from the liquid transfer pump to flow to the liquid storage unit.

The drive unit may be configured to be rotatable in an opposite direction to a direction of rotation during discharging of the liquid.

The first flow channel may be constituted by a first tube and the second flow channel may be constituted by a second tube, and the flexible tube, the first tube, and the second tube may be configured to be replaceable.

The flow channel switching unit may be a pinch valve.

In order to accomplish the above-mentioned objective, a liquid discharge control method according to an embodiment of the present technology includes causing a liquid transferred from a liquid transfer pump to flow out to a first flow channel on a discharge side at a predetermined rate, the liquid transfer pump including a flexible tube that allows a liquid to circulate therethrough, a pressing unit that presses the flexible tube from an outer circumferential surface side and feeds the liquid from one end to another end of the flexible tube, and a drive unit that rotationally drives the pressing unit, and then causing the liquid to flow out to a second flow channel different from the first flow channel until the drive unit moves to a position where the drive unit is located when the liquid starts to flow out to the first flow channel.

With such a configuration, the discharge is performed so that the position of the flexible tube that is pressed by the pressing unit is constantly the same position every time. The discharge rate at every time can be thus controlled with high accuracy.

In order to accomplish the above-mentioned objective, a filling method according to an embodiment of the present technology includes: discharging a liquid transferred from a liquid transfer pump into a container so as to fill the container through a first flow channel at a predetermined rate, the liquid transfer pump including a flexible tube that allows a liquid to circulate therethrough, a pressing unit that presses the flexible tube from an outer circumferential surface side and feeds the liquid from one end to another end of the flexible tube, and a drive unit that rotationally drives the pressing unit, and then causing the liquid to flow out to a second flow channel different from the first flow channel until the drive unit moves to a position where the drive unit is located when the liquid starts to flow out to the first flow channel.

With such a configuration, the discharge is performed so that the position of the flexible tube that is pressed by the pressing unit is constantly the same position every time. The container can be thus filled with the liquid with high accuracy and a constant discharge rate.

### BRIEF DESCRIPTION OF DRAWINGS

FIG. 1 A schematic view describing a configuration of a liquid discharge apparatus.

FIG. 2 A schematic view describing a configuration of a liquid transfer pump that is a part of the liquid discharge apparatus.

FIG. 3 A schematic view describing a liquid flow direction in the liquid discharge apparatus.

FIG. 4 A diagram describing a flow of an operation in the liquid discharge apparatus.

FIG. 5 A flow diagram of a liquid discharge control method in the liquid discharge apparatus.

### MODE(S) FOR CARRYING OUT THE INVENTION

Hereinafter, the present technology will be described with reference to the drawings.

[Configuration of Liquid Discharge Apparatus]

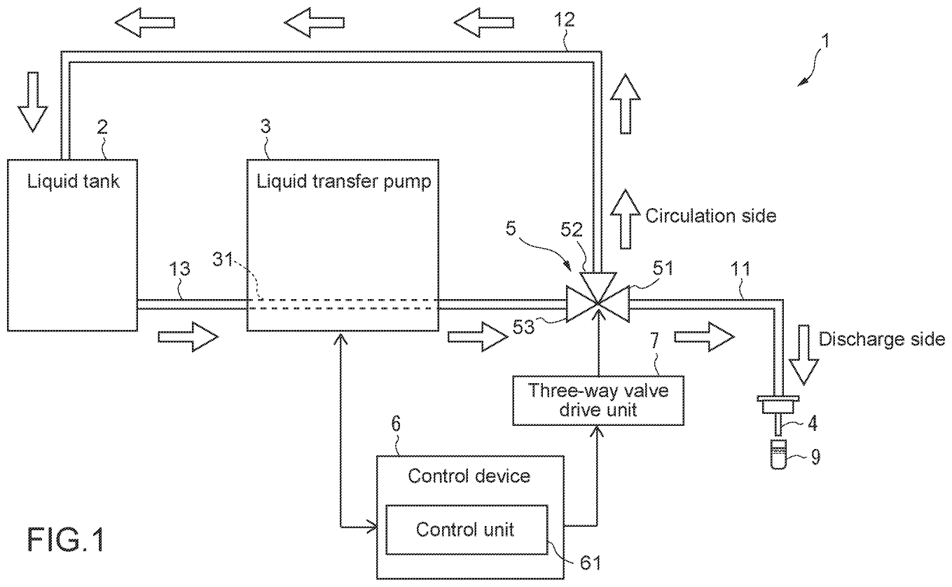

FIG. 1 is a schematic view describing an overall configuration of a liquid discharge apparatus. In FIG. 1, the hollow arrow indicates a liquid flow.

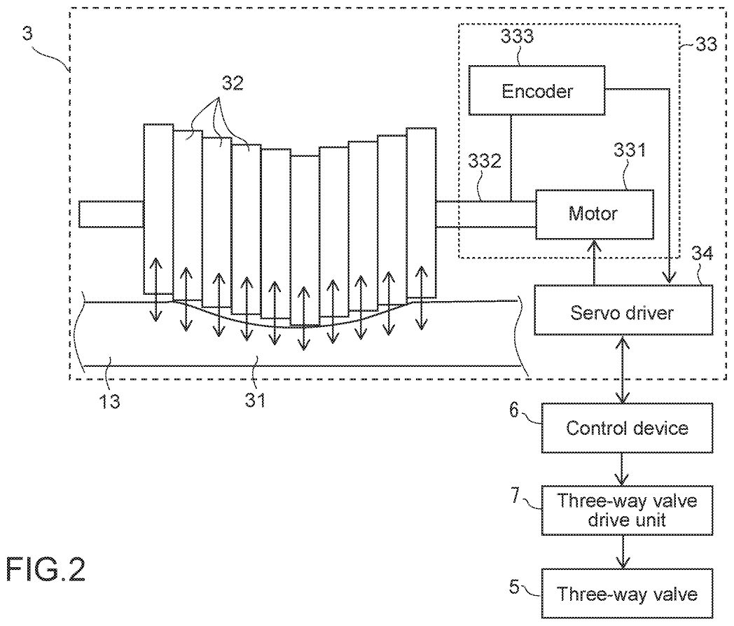

FIG. 2 is a schematic view of a portion of the liquid discharge apparatus. FIG. 2 is a diagram mainly describing a structure of a liquid transfer pump constituting a part of the liquid discharge apparatus.

As shown in FIG. 1, a liquid discharge apparatus 1 includes a liquid tank 2, a liquid transfer pump 3, a first tube 11, a second tube 12, a discharge head 4, a three-way valve 5, a control device 6, and a three-way valve drive unit 7.

The liquid discharge apparatus 1 is configured so that a liquid such as a liquid medicine stored in the liquid tank 2 can be transferred by the liquid transfer pump 3 and discharged into a container 9 from the discharge head 4. It can also be said that the liquid discharge apparatus 1 is a filling apparatus that dispenses a liquid into the container 9 so as to fill the container 9.

It should be noted that although an example of filling the container with the liquid will be described here, a discharge destination of a liquid discharged from the liquid discharge apparatus 1 is not limited to the container. For example, the present technology can also be applied to an embodiment in which a liquid as a painting liquid is discharged to an object to be painted from the liquid discharge apparatus, for example. The liquid discharge apparatus 1 is configured to be capable of dispensing a liquid at a predetermined rate. For example, the discharge rate can be set to be 0.01 ml/min to 100 ml/min. The liquid discharge apparatus 1 according to the present embodiment is capable of high-accuracy dispensing even at a low discharge rate, in particular, 0.01 ml/min to 1.0 ml/min. It is effective.

The liquid tank 2 is a liquid storage unit that stores a liquid discharged into the container 9 from the liquid discharge apparatus 1.

The liquid transfer pump 3 transfers a liquid stored in the liquid tank 2 to the first tube 11 or the second tube 12. As shown in FIG. 2, the liquid transfer pump 3 includes a third tube 13, a plurality of eccentric cams 32, a servo motor 33, and a servo driver 34. Hereinafter, each configuration will be described.

The third tube 13 has a hollow elongated shape and its hollow portion serves as a flow channel. The third tube 13 is, at one end, connected to the liquid tank 2 and is, at the other end, connected to the three-way valve 5. At least a portion of the third tube 13, which is pressed by eccentric cams 32 to be described later, has flexibility. Here, a portion of the third tube 13, which is pressed by the eccentric cams 32, will be referred to as a flexible tube 31. A liquid supplied from the liquid tank 2 can circulate through the flexible tube 31.

The flexible tube 31 is made from an elastic material that is pressed and deformed from its outer circumferential surface side by the eccentric cams 32 and returns to the original shape when it is released from the press. A liquid supplied into the flexible tube 31 is fed from the one end to the other end in such a manner that the plurality of eccentric cams 32 operate, the eccentric cams 32 press the flexible tube 31 in order from the one end to the other end of the flexible tube 31 and squeeze the flexible tube 31 in one direction.

The servo motor 33 has a motor 331 as a drive unit and an encoder 333 as a detection unit.

The motor 331 has a rotation shaft 332. The motor 331 is rotationally driven, supplied with a driving current based on a motor control signal (typically, a pulse signal) according to its operation from the servo driver 34. The plurality of eccentric cams 32 is rotationally driven along with the rotational driving so as to press the flexible tube 31. The motor control signal is an operation command signal for the motor. Here, a rotation of the motor 331 when the liquid transfer pump 3 transfers a liquid from the liquid tank 2 to the first tube 11 or the second tube 12 will be referred to as a positive rotation. A rotation in a direction of rotation opposite to this positive rotation will be referred to as a reverse rotation. The positive rotation direction is a direction of rotation during liquid discharge or liquid circulation. Hereinafter, the “positive rotation” will be simply referred to as a “rotation”.

The encoder 333 is a rotation position detector that is a detection unit that detects a change in position of the motor 331. The encoder 333 is coupled with the rotation shaft 332 of the motor 331. The encoder 333 outputs a detection signal, for example, a number of pulses (pulse signals) depending on an amount of rotation. An output signal (referred to as a detection signal in some cases) from the encoder includes rotation position information (angle-of-rotation information), rotation speed information, and the like of the motor 331. For example, a type of detector that outputs A-phase, B-phase, and Z-phase pulse signals can be used for the encoder 333. The A-phase and B-phase pulse signals are proportional to the speed. The B-phase outputs a pulse with a phase difference of 90 degrees with respect to A-phase, and a direction of rotation is determined on the basis of a B-phase signal level during a rise of the A-phase pulse. The Z-phase pulse signal is a pulse signal generated every time the motor rotates once.

The encoder 333 generates a detection signal indicating a detected operation of the motor 331 and sends the detection signal to the servo driver 34.

The plurality of eccentric cams 32 rotationally operates along with the motion of the rotation shaft 332 of the motor 331 and presses the flexible tube 31. The plurality of eccentric cams 32 is a pressing unit that presses the flexible tube 31. The eccentric cams 32 press the flexible tube 31 in order from the one end to the other end so as to be capable of feeding a liquid supplied into the flexible tube 31 from the one end to the other end.

The servo driver 34 receives from the control device 6 a motor control signal (operation command signal of the motor) related to the operation of the motor 331, and supplies a driving current to the motor 331 so that the operation of the motor 331 follows the command value.

Moreover, the servo driver 34 receives a detection signal output from the encoder 333. The servo driver 34 performs servo control related to driving of the motor 331 on the basis of the motor control signal and the detection signal from the encoder 333. That is, the servo driver 34 calculates the command value related to the operation of the motor 331 on the basis of the motor control signal and the detection signal from the encoder 333, and supplies the motor 331 with a driving current so that the operation of the motor 331 follows the command value.

The first tube 11 and the second tube 12 have a hollow elongated shape and the hollow portions serve as flow channels.

The first tube 11 constitutes a first flow channel through which a liquid transferred from the liquid transfer pump 3 flows. One end of the first tube 11 is provided with the discharge head 4. The other end of the first tube 11 is connected to the three-way valve 5. A liquid transferred from the liquid transfer pump 3 and flowing into the first tube 11 is discharged from the discharge head 4 to the container 9.

The second tube 12 constitutes a second flow channel through which a liquid transferred from the liquid transfer pump 3 flows. The first flow channel and the second flow channel are different from each other. One end of the second tube 12 is connected to the liquid tank 2. The other end of the second tube 12 is connected to the three-way valve 5. A liquid transferred from the liquid transfer pump 3 and flowing into the second tube 12 is supplied to the liquid tank 2.

In this manner, the first tube 11 functions as a tube on a discharge side and the second tube 12 functions as a tube on a circulation side.

Wetted portions of the liquid discharge apparatus 1, with which a liquid is in direct contact, are respective inner portions of the first tube 11, the second tube 12, and the third tube 13 with the flexible tube 31. In the liquid discharge apparatus 1, these tubes are configured to be replaceable and disposable. The tube replacement makes washing and sterilization processes of the wetted portions for the changeover unnecessary. It can enhance workability and safety. Moreover, it can avoid liquid contamination possibility.

The three-way valve 5 is a flow channel switching unit that changes the flow channel through which a liquid transferred from the liquid transfer pump 3 flows into the first tube 11 or the second tube 12. The three-way valve 5 is a flow channel open/close valve.

The three-way valve 5 includes an inflow portion 53 in which a liquid transferred from the liquid transfer pump 3 flows, a first outflow portion 51 that allows a liquid transferred from the liquid transfer pump 3 via the inflow portion 53 to flow out to the first tube 11, and a second outflow portion 52 that allows a liquid transferred from the liquid transfer pump 3 via the inflow portion 53 to flow out to the second tube 12.

In the three-way valve 5, the three-way valve drive unit 7 controls open/close of the first outflow portion 51 and the second outflow portion 52 on the basis of a control signal from a control unit 61 of the control device 6 to be described later. It should be noted that the inflow portion 53 is constantly open and no valves are provided.

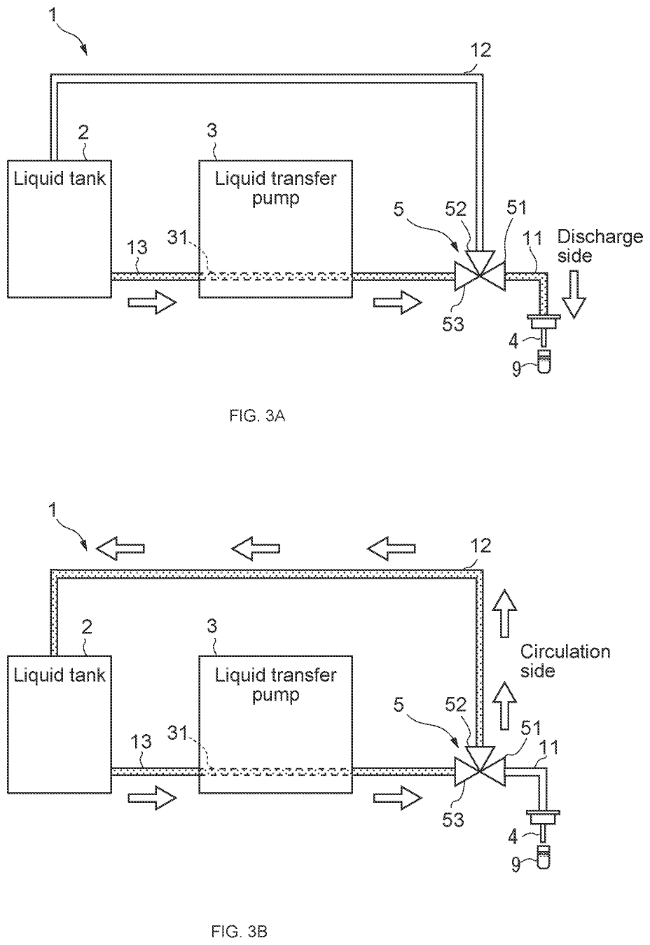

FIG. 3 is a schematic view describing a liquid flow direction in the liquid discharge apparatus 1.

As shown in FIG. 3(A), when the first outflow portion 51 is opened and the second outflow portion 52 is closed in the three-way valve 5, a liquid transferred by the liquid transfer pump 3 flows out to the first tube 11 on the discharge side. The liquid flowing to the first tube 11 is discharged into the container 9 so as to fill the container 9.

As shown in FIG. 3(B), when the second outflow portion 52 is opened and the first outflow portion 51 is closed in the three-way valve 5, a liquid transferred by the liquid transfer pump 3 flows out to the second tube 12 on the circulation side. The liquid flowing to the second tube 12 flows into the liquid tank 2.

Typically, a solenoid pinch valve can be used as the three-way valve 5, though not limited thereto. Since the wetted portions of the pinch valve can be only tubes, it can keep the flow channels constantly clean. It is thus more favorable.

The three-way valve drive unit 7 controls the valve open/close state of the three-way valve 5 on the basis of a valve control signal sent from the control unit 61 of the control device 6.

The control device 6 has the control unit 61.

An input operation unit (not shown) such as a touch panel and a keyboard is connected to the control device 6. For example, the worker of the liquid discharge apparatus 1 inputs various commands such as a rotation speed, r.p.m. of the motor associated with a discharge rate per one time, discharge operation start and operation stop, a change in direction of rotation from the input operation unit. The various types of operation command information are sent to the control device 6.

The control unit 61 controls the valve open/close state of the three-way valve 5. The control unit 61 may further control the operation of the motor 331. For example, a personal computer can be used as the control device 6. The control unit 61 includes a central processing unit (CPU) and the like. The CPU executes a control program associated with the control of the open/close state of the three-way valve 5 or the operation control of the motor 331.

The control program associated with the control of the open/close state of the three-way valve 5 or the operation control of the motor 331 can be prestored in a hard disk or read only memory (ROM) as a built-in recording medium of the control device 6. Alternatively, the control program can be stored in a removable recording medium driven by a drive. Such a removable recording medium can be provided as so-called package software. Examples of the removable recording medium can include a flexible disk, a compact disc read-only memory (CD-ROM), a magneto-optical (MO) disc, a digital versatile disc (DVD), a magnetic disk, and a semiconductor memory. It should be noted that the program can be downloaded to a control device via a communication network and installed in the built-in hard disk other than being installed in a computer from the removable recording medium. The control program will be described later.

On the basis of the various types of command information received, the control unit 61 sends the motor control signal related to the operation of the motor 331 to the servo driver 34. In addition, the control unit 61 receives a detection signal of the encoder 333 from the servo driver 34 and generates a valve control signal for controlling the valve open/close state of the three-way valve 5 on the basis of the detection signal.

To be specific, the control unit 61 sends to the servo driver 34 a motor control signal for discharge for transferring a liquid at a predetermined rate so that the container 9 is filled with the liquid at the predetermined rate. The “predetermined rate” is a discharge rate per one time that is discharged from the liquid discharge apparatus 1. Hereinafter, the “liquid at the predetermined rate” will be referred to as a “liquid for discharge” in some cases. Moreover, a process in which a liquid is discharged to the container 9 will be referred to as a discharge process.

In addition, as shown in FIG. 3(A), the control unit 61 sends a valve control signal to the three-way valve drive unit 7 so as to control the three-way valve 5 so that the first outflow portion 51 is opened and the second outflow portion 52 is closed while the liquid for discharge is transferred by the liquid transfer pump 3 on the basis of the motor control signal for discharge.

Accordingly, a liquid transferred from the liquid transfer pump 3 for discharge flows to the first tube 11. A liquid flowing through the first tube 11 is discharged to the container 9 at a predetermined discharge rate. Here, a position of the motor 331 at the outflow start of the liquid in one discharge process will be referred to as a reference position and a position of the motor 331 at the outflow end will be referred to as an end position.

Next, the control unit 61 sends to the servo driver 34 a motor control signal for phase adjustment for the motor 331 to rotate and return to the reference position from the end position after the liquid for discharge is transferred to the first tube 11.

In addition, after the liquid for discharge is transferred to the first tube 11, the control unit 61 sends to the three-way valve drive unit 7 a valve control signal for controlling the three-way valve 5 so that the liquid transferred by the liquid transfer pump 3 flows out to the second tube 12 as shown in FIG. 3(B) on the basis of the motor control signal for phase adjustment. This valve control signal is for controlling the three-way valve 5 so that the second outflow portion 52 is opened and the first outflow portion 51 is closed in the three-way valve 5.

Accordingly, on the basis of the motor control signal for phase adjustment, a liquid transferred from the liquid transfer pump 3 flows to the second tube 12 and the motor 331 rotates and returns to the reference position from the end position. The movement of the motor 331 to the reference position from the end position will be referred to as a phase adjustment. The liquid flowing to the second tube 12 is caused to circulate to the liquid tank 2. This process in which the liquid circulates will be referred to as a circulation process. It should be noted that the phase of the motor refers to a positional relationship between the eccentric cams 32 that are the pressing unit and the motor 331. The “phase adjustment” described above is rotationally moving the motor 331 so that the phase of the motor at the outflow end of the liquid in the one discharge process becomes the phase of the motor at the outflow start.

In the liquid discharge apparatus 1, the discharge process and the circulation process are alternately repeated. In the one discharge process, a single container is filled with a liquid.

In this manner, the control unit 61 sends to the servo driver 34 the motor control signal for discharge and the motor control signal for phase adjustment.

In addition, the control unit 61 generates a valve control signal of controlling the valve open/close state of the three-way valve 5 so that a liquid transferred from the liquid transfer pump 3 flows to the first tube 11 on the basis of the motor control signal for discharge and a liquid transferred from the liquid transfer pump 3 flows to the second tube 12 on the basis of the motor control signal for phase adjustment, and sends the valve control signal to the three-way valve drive unit.

Since the motor operation and the valve open/close state of the three-way valve are controlled as described above, the motor 331 is positioned at the reference position at the start of the next discharge. By performing this control during dispensing, the motor 331 is constantly positioned at the reference position at the start of the discharge process in every discharge process. Thus, in each discharge process, positions of the flexible tube 31, which are pressed by the eccentric cams 32, are constantly the same positions.

Here, the inner diameter of the flexible tube is not necessarily uniform. Moreover, it is difficult to work the eccentric cams to each have a dimension identical to a reference dimension, and the respective eccentric cams may vary in dimension.

In the present embodiment, even if the inner diameter of the flexible tube is not uniform or the plurality of eccentric cams vary in dimension, the positions of the flexible tube, which are pressed by the eccentric cams, are controlled to be constantly the same positions in every discharge process. Also, each eccentric cam presses constantly the same position of the flexible tube. Therefore, the discharge rate at every time can be stably controlled to be a constant rate. In this way, the liquid discharge apparatus 1 is capable of discharging with high accuracy.

As described above, the motor control signal related to the operation of the motor 331 includes the motor control signal for discharge and the motor control signal for phase adjustment. The three-way valve 5 is controlled so that the liquid transferred by the liquid transfer pump 3 flows out to the first tube 11 on the discharge side on the basis of the motor control signal for discharge and the liquid transferred by the liquid transfer pump 3 flows out to the second tube 12 on the circulation side on the basis of the motor control signal for phase adjustment. The liquid flowing out to the second tube 12 is returned to the liquid tank 2.

In this manner, during the phase adjustment, the liquid transferred by the liquid transfer pump 3 is returned to the liquid tank 2 and the liquid circulates. Therefore, the liquid is hardly wasted and the liquid utilization efficiency can be enhanced.

The control of the above-mentioned three-way valve 5 will be described more specifically.

The motor 331 of the liquid transfer pump 3 rotationally drives the eccentric cams 32 on the basis of a driving current generated by the servo driver 34 on the basis of the motor control signal. The motor control signal is typically a pulse signal.

The number of pulses of the motor control signal for discharge that corresponds to the discharge rate per one time is defined as m (m>0). The motor control signal for discharge that corresponds to the discharge rate per one time will be referred to as a pulse signal for discharge in some cases. Hereinafter, the motor control signal for phase adjustment will be referred to as a pulse signal for phase adjustment in some cases.

The number of pulses of the pulse signal that is required for the motor 331 to rotate once is defined as n (n>0).

In a case where n<m, the three-way valve 5 is controlled by the three-way valve drive unit 7 on the basis of the valve control signal sent from the control unit 61 as follows.

That is, as shown in FIG. 3(A), the three-way valve 5 is controlled so that the first outflow portion 51 is opened and the second outflow portion 52 is closed. Accordingly, a liquid transferred from the liquid transfer pump 3 on the basis of the pulse signal for discharge with the number of pulses m is made to flow to the first tube 11.

After the liquid transfer based on the pulse signal for discharge, the three-way valve 5 is controlled so that the first outflow portion 51 is closed and the second outflow portion 52 is opened as shown in FIG. 3(B). Accordingly, a liquid transferred from the liquid transfer pump 3 on the basis of the pulse signal with the number of pulses b satisfying m=an+b (a denotes one or more integers, b>0, n>b) is made to flow to the second tube 12. The pulse signal with the number of pulses b is the pulse signal for phase adjustment.

In a case where n>m, the three-way valve 5 is controlled by the three-way valve drive unit 7 on the basis of the valve control signal sent from the control unit 61 as follows.

That is, as shown in FIG. 3(A), the three-way valve 5 is controlled so that the first outflow portion 51 is opened and the second outflow portion 52 is closed. Accordingly, a liquid transferred from the liquid transfer pump 3 on the basis of the pulse signal for discharge with the number of pulses m is made to flow to the first tube 11.

After the liquid transfer based on the pulse signal for discharge, the three-way valve 5 is controlled so that the first outflow portion 51 is closed and the second outflow portion 52 is opened as shown in FIG. 3(B). Accordingly, a liquid transferred from the liquid transfer pump 3 on the basis of the pulse signal with the number of pulses c satisfying c=n−m (c>0) is made to flow to the second tube 12. The pulse signal with the number of pulses c is the pulse signal for phase adjustment.

An example of the control of the valve open/close state of the three-way valve 5 will be described taking specific numeric values.

An example in which the motor 331 that rotates once at 500 pulses is used as the driving source and the discharge rate per one time is controlled at the amount of rotation at 300 pulses, where n<m, will be shown. When the motor 331 rotates once, the encoder 333 outputs 500 pulses as the A-phase and B-phase output and 1 pulse as the Z-phase output for example as detection results.

In this case, the liquid transfer pump 3 is controlled to operate on the basis of the pulse signal for phase adjustment with 200 pulses after operating on the basis of the pulse signal for discharge with 300 pulses. The three-way valve 5 is controlled in accordance with the valve control signal so that a liquid transferred from the liquid transfer pump 3 flows to the first tube 11 on the basis of the pulse signal for discharge and a liquid transferred from the liquid transfer pump 3 flows to the second tube 12 on the basis of the pulse signal for phase adjustment.

In other words, the discharge rate per one time is equivalent to a 0.6-rotation of the motor, and in the discharge process, the first outflow portion 51 is set to be opened and the second outflow portion 52 is set to be closed. In order for the motor 331 to return to the reference position from the end position after the discharge process, it is sufficient to perform a 0.4-rotation, and in a period until it is returned to this reference position, i.e., in a phase adjustment process, the first outflow portion 51 is set to be closed and the second outflow portion 52 is set to be opened. On the basis of the pulse signal for phase adjustment, the motor 331 returns to the reference position, and in a discharge process based on a next pulse signal for discharge, the rotation of the motor 331 starts from the reference position.

An example in which the motor 331 that rotates once at 500 pulses is used as the driving source and the discharge rate per one time is controlled at the amount of rotation at 1300 pulses, where n>m, will be shown. When the motor 331 rotates once, the encoder 333 outputs 500 pulses as the A-phase and B-phase output and 1 pulse as the Z-phase output for example as detection results.

In this case, the liquid transfer pump 3 is controlled to operate on the basis of the pulse signal for phase adjustment with 200 pulses after operating on the basis of the pulse signal for discharge with 1300 pulses. The three-way valve 5 is controlled in accordance with the valve control signal so that a liquid transferred from the liquid transfer pump 3 flows to the first tube 11 on the basis of the pulse signal for discharge and a liquid transferred from the liquid transfer pump 3 flows to the second tube 12 on the basis of the pulse signal for phase adjustment.

In other words, the discharge rate per one time is equivalent to a 2.6-rotation of the motor 331, and in the discharge process, the first outflow portion 51 is set to be opened and the second outflow portion 52 is set to be closed. In order for the motor 331 to return to the reference position from the end position after the discharge process, it is sufficient to perform a 0.4-rotation, and in a period until it is returned to this reference position, i.e., in a phase adjustment process, the first outflow portion 51 is set to be closed and the second outflow portion 52 is set to be opened. By driving based on the pulse signal for phase adjustment, the motor 331 returns to the reference position, and in a discharge process based on a next pulse signal for discharge, the rotation of the motor 331 starts from this reference position.

In general, it has been considered that pumps with disposable tubes are inferior in liquid-feeding accuracy and dispensing accuracy and they are not suitable for the use in applications where high repeatability and reproducibility are desired. In this regard, the liquid discharge apparatus 1 using the pump with the tube according to the present embodiment is controlled so that the rotation start position of the motor is constantly the same position (reference position) at every discharge time. Accordingly, the discharge rate at every time can be stably controlled to be a constant rate, and the dispensing accuracy can be increased to about ±1% that is substantially equivalent to that of a high-precision volumetric pump.

Thus, in the liquid discharge apparatus 1 according to the present embodiment, the wetted portions are disposable while discharging with high accuracy is realized. Moreover, since the pinch valve is used as the three-way valve, the disposable wetted portions are the tubes. Since the wetted portions are disposable, the washing and sterilization processes of the wetted portions become unnecessary. Thus, the working time can be greatly shortened, the worker safety can be ensured, and the liquid contamination possibility can be greatly reduced. It should be noted that the three-way valve does not necessarily need to be the pinch valve, and a three-way valve having wetted portions may be employed and the three-way valve may be replaced. However, in view of reduction of the number of components to be replaced, workability, and the like, it is more favorable to use the pinch valve.

In addition, in the liquid discharge apparatus 1, the liquid transferred during the phase adjustment can be returned to the liquid tank 2. Therefore, the waste of the liquid is saved, and the liquid utilization efficiency is enhanced. It should be noted that a supply destination to which the liquid transferred during the phase adjustment is supplied does not need to be the liquid tank, and only needs to be a location different from the discharge destination via the first tube 11 on the discharge side. However, in view of the liquid utilization efficiency as described above, it is more favorable to cause the liquid to circulate in the liquid tank.

Moreover, in the liquid discharge apparatus 1, the liquid circulation and the liquid discharge are switched through the three-way valve 5. Therefore, it is unnecessary to move the discharge head during the phase adjustment, time loss is avoided during mass production, and liquid dispensing can be performed continuously.

Moreover, the motor 331 is configured to be rotatable in an opposite direction to the direction of rotation during the liquid discharge. Accordingly, after the dispensing, the liquid remaining in the tube can be returned to the liquid tank by rotating the motor 331 reversely, and the liquid utilization efficiency is enhanced.

[Liquid Discharge Control Method and Filling Method]

A liquid discharge control method and a filling method using the liquid discharge apparatus 1 will be described. Here, a filling method of filling a container with a liquid discharged by a liquid discharge control method will be shown as an example.

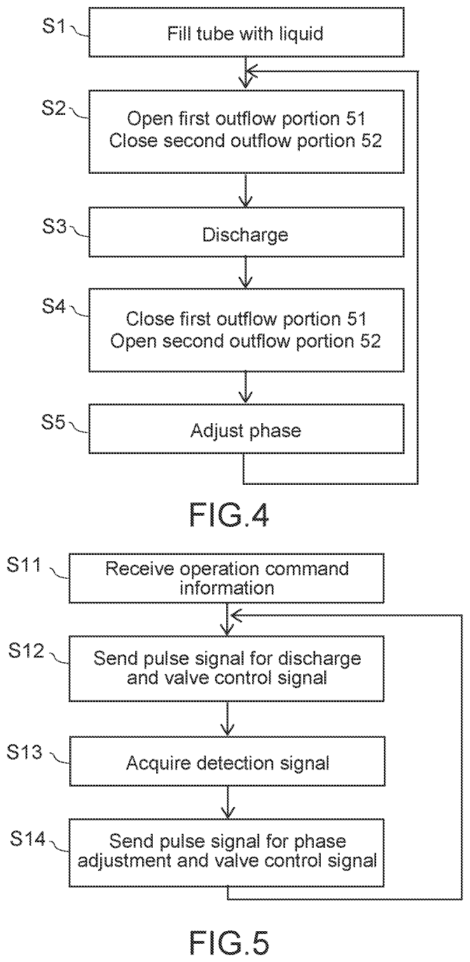

FIG. 4 is a flow diagram describing a flow of an operation by the liquid discharge apparatus 1.

FIG. 5 is a flow diagram of the liquid discharge control method by the control unit 61 of the control device 6.

Hereinafter, the description will be given with reference to FIGS. 4 and 5.

As shown in FIG. 4, when the dispensing process by the liquid discharge apparatus 1 is started, first of all, the first outflow portion 51 is closed and the second outflow portion 52 is opened, and the second tube 12 and the third tube 13 are filled with a liquid. Thereafter, the second outflow portion 52 is closed and the first outflow portion 51 is opened, and the first tube 11 is filled with a liquid (S1). Moreover, for example, operation command information input by a worker is sent to the control device 6.

As shown in FIG. 5, the control unit 61 of the control device 6 receives the operation command information (S11).

On the basis of the received operation command information, the control unit 61 sends to the three-way valve drive unit 7 a valve control signal to set the first outflow portion 51 to be opened and the second outflow portion 52 to be closed in the three-way valve 5, and sends to the servo motor 33 a pulse signal for discharge (S12).

The three-way valve drive unit 7 controls the valve open/close state of the three-way valve 5 on the basis of the valve control signal. The servo motor 33 receives the pulse signal for discharge, and supplies the motor 331 with a driving current so that the operation of the motor 331 follows its command value.

Accordingly, as shown in FIG. 4, in the three-way valve 5, the first outflow portion 51 is opened and the second outflow portion 52 is closed (S2). The motor 331 of the liquid transfer pump 3 is driven on the basis of the pulse signal for discharge and a liquid is transferred. A liquid transferred from the liquid transfer pump 3 flows to the first tube 11, and is discharged into the container 9 from the discharge head 4 attached to one end of the first tube 11 (S3) so as to fill the container 9 (see FIG. 3(A)).

As shown in FIG. 5, the control unit 61 acquires a detection signal detected by the encoder (S13).

The control unit 61 detects the end of the discharge process on the basis of the acquired detection signal. Thereafter, the control unit 61 sends to the three-way valve drive unit 7 a valve control signal to set the second outflow portion 52 to be opened and the first outflow portion 51 to be closed in the three-way valve 5. In addition, on the basis of the detection signal, the control unit 61 generates a pulse signal for phase adjustment for rotationally driving the motor so that the position (end position) of the motor at the end of one discharge returns to the reference position, and sends the pulse signal for phase adjustment to the servo motor 33 (S14).

The three-way valve drive unit 7 controls the valve open/close state of the three-way valve 5 on the basis of the valve control signal. The servo motor 33 receives the pulse signal for phase adjustment and supplies the motor 331 with a driving current so that the operation of the motor 331 follows its command value.

Accordingly, as shown in FIG. 4, in the three-way valve 5, the second outflow portion 52 is opened and the first outflow portion 51 is closed (S4). The motor 331 of the liquid transfer pump 3 is driven on the basis of the pulse signal for phase adjustment, the phase adjustment is performed, and the motor 331 returns to the reference position (S5). On the basis of the pulse signal for phase adjustment, a liquid transferred from the liquid transfer pump 3 flows to the second tube 12 and returns to the liquid tank 2 connected to one end of the second tube 12 (see FIG. 3(B)).

As shown in FIG. 4, after the phase adjustment, the process returns to S2, discharge process and phase adjustment process are repeatedly performed, and dispensing is performed continuously in the liquid discharge apparatus 1.

In the present embodiment, since the rotation start position of the motor in each discharge is controlled to be constantly the same position (reference position), the discharge rate at every time can be stably controlled to be a constant rate. Thus, each of a plurality of containers 9 can be filled with a liquid at a constant discharge rate with high accuracy, and containers with little variation in liquid filling rate can be obtained.

After the dispensing process ends, each of the first outflow portion 51 and the second outflow portion 52 of the three-way valve 5 is switched to the open state, and the liquid transfer pump 3 is rotated reversely, such that the liquid in the tubes can be entirely returned to the liquid tank 2. Accordingly, a waste liquid can be reduced, and the liquid utilization efficiency is enhanced.

[Control Program]

The control program of the open/close state of the three-way valve 5 controls the three-way valve 5 as follows.

That is, the three-way valve 5 is controlled so that the flow channel to which the liquid at the predetermined rate, which is transferred from the liquid transfer pump 3, flows out is the first tube (first flow channel) 11. Then, the three-way valve 5 is controlled so that the flow channel to which a liquid transferred from the liquid transfer pump 3 flows out is the second tube (second flow channel) 12 until the motor 331 moves to the position (reference position) at the outflow start of the liquid to the first tube 11 after the liquid at the predetermined rate flows out.

The control program of the operation of the motor 331 controls the motor 331 as follows.

That is, the motor 331 is controlled so that the liquid at the predetermined rate is made to flow out in accordance with a motor control signal for discharge when the flow channel to which a liquid transferred from the liquid transfer pump 3 is made to flow out is the first tube 11. Then, the motor 331 is controlled so that the motor 331 moves to the reference position from the end position in accordance with the motor control signal for phase adjustment when the flow channel to which a liquid transferred from the liquid transfer pump 3 is made to flow out is the second tube 12 after the liquid at the predetermined rate flows out.

Embodiments of the present technology are not limited to the above-mentioned embodiment and various changes can be made without departing from the gist of the present technology.

The example in which the servo motor is used as the liquid transfer pump according to the above-mentioned embodiment has been described as an example, though not limited thereto. For example, a stepping motor may be used. In this case, the detector (detection unit) such as the encoder that detects motor rotation information and the like can be provided.

Moreover, the example in which the flexible tube is arranged in a straight-line form in the liquid transfer pump has been described in the above-mentioned embodiment as an example, though not limited thereto. For example, an embodiment in which the liquid transfer pump has a flexible tube bent in a U-shape and this flexible tube is pushed by a roller serving as the plurality of pressers from one end at its outer peripheral portion to the other end for transferring a liquid in the tube may be employed.

Also in such an embodiment, by performing phase adjustment, constantly the same position of the flexible tube can be pressed for transferring a liquid for each discharge as in the above-mentioned embodiment. Thus, the liquid can be discharged at a constantly stable discharge rate.

Moreover, for example, in the above-mentioned liquid discharge apparatus, a flow rate detection unit and a weight detector may be further provided. The flow rate detection unit detects an amount of liquid that is transferred from the liquid transfer pump and the flows in the first tube, i.e., a flow rate that is the discharge rate. The weight detector is disposed below the container to be filled with a liquid and detects container weight before and after filling.

On the basis of a flow rate detection result of the flow rate detection unit and a desired discharge rate, the control unit may generate a motor control signal. A filling rate may be calculated by measuring container weight before and after filling through the weight detector and the control unit may generate a motor control signal on the basis of the filling rate.

Accordingly, even if a tube has been replaced and an inner diameter variation between tubes different from each other is different, a liquid can be discharged at a constantly stable predetermined rate.

For example, an electromagnetic flowmeter, a Coriolis flowmeter, or the like can be used as the flow rate detection unit, and a contactless flowmeter is more favorably used in view of liquid contamination prevention. Moreover, a measurement method using the weight detector is a contactless measurement method. Therefore, it is favorable in view of liquid contamination prevention.

It should be noted that the present technology may also take the following configurations.

(1) A liquid discharge apparatus, including:
a liquid transfer pump including
   a flexible tube that allows a liquid to circulate therethrough, the liquid being supplied from a liquid storage unit that stores the liquid,
   a pressing unit that presses the flexible tube from an outer circumferential surface side and feeds the liquid from one end to another end of the flexible tube, and
   a drive unit that rotationally drives the pressing unit;
a first flow channel to which a liquid transferred from the liquid transfer pump flows out;
a second flow channel to which a liquid transferred from the liquid transfer pump flows out, the second flow channel being different from the first flow channel; and
a flow channel switching unit having a flow channel to which a liquid transferred from the liquid transfer pump flows out as the first flow channel or the second flow channel, the flow channel switching unit being controlled to use, as the first flow channel, a flow channel to which a liquid for discharge that is transferred from the liquid transfer pump flows out and use, as the second flow channel, a flow channel to which a liquid transferred from the liquid transfer pump flows out until the drive unit moves to a position after the liquid for discharge is transferred, the position being a position where the drive unit is located when the liquid for discharge starts to flow out.

(2) The liquid discharge apparatus according to (1), in which
the drive unit of the liquid transfer pump is driven on the basis of a pulse signal, and
assuming that a number of pulses of the pulse signal for discharge, which is for transferring the liquid for discharge, is defined as m (m>0) and a number of pulses of the pulse signal, which is necessary for the drive unit to rotate once, is defined as n (n>0),
in a case where n<m,
the flow channel switching unit is controlled to use, as the first flow channel, a flow channel to which a liquid transferred from the liquid transfer pump driven on the basis of the pulse signal for discharge flows out and use, as the second flow channel, a flow channel to which a liquid transferred from the liquid transfer pump driven on the basis of the pulse signal with a number of pulses b satisfying m=an+b (a denotes one or more integers, b>0, n>b) flows out after the liquid for discharge is transferred, and
in a case where n>m,
the flow channel switching unit is controlled to use, as the first flow channel, a flow channel to which a liquid transferred from the liquid transfer pump driven on the basis of the pulse signal for discharge flows out and use, as the second flow channel, a flow channel to which a liquid transferred from the liquid transfer pump driven on the basis of a pulse signal with a number of pulses c satisfying c=n−m flows out after the liquid for discharge is transferred.

(3) The liquid discharge apparatus according to (2), further including
a control unit that controls the flow channel switching unit.

(4) The liquid discharge apparatus according to (3), further including
a detection unit that detects a change in position of the drive unit, in which
the control unit controls the flow channel switching unit on the basis of a detection result of the detection unit.

(5) The liquid discharge apparatus according to (3) or (4), in which
the control unit further controls an operation of the drive unit, and
the control unit
causes, in a case where n<m, the drive unit to operate on the basis of the pulse signal for discharge and then operate on the basis of the pulse signal with the number of pulses b, and
causes, in a case where n>m, the drive unit to operate on the basis of the pulse signal for discharge and then operate on the basis of the pulse signal with the number of pulses c.

(6) The liquid discharge apparatus according to any one of (1) to (5), in which
the second flow channel causes a liquid transferred from the liquid transfer pump to flow to the liquid storage unit.

(7) The liquid discharge apparatus according to any one of (1) to (6), in which
the drive unit is configured to be rotatable in an opposite direction to a direction of rotation during discharging of the liquid.

(8) The liquid discharge apparatus according to any one of (1) to (7), in which
the first flow channel is constituted by a first tube and the second flow channel is constituted by a second tube, and
the flexible tube, the first tube, and the second tube are configured to be replaceable.

(9) The liquid discharge apparatus according to any one of (1) to (8), in which
the flow channel switching unit is a pinch valve.

(10) A liquid discharge control method, including
causing a liquid transferred from a liquid transfer pump to flow out to a first flow channel on a discharge side at a predetermined rate, the liquid transfer pump including a flexible tube that allows a liquid to circulate therethrough, a pressing unit that presses the flexible tube from an outer circumferential surface side and feeds the liquid from one end to another end of the flexible tube, and a drive unit that rotationally drives the pressing unit, and then causing the liquid to flow out to a second flow channel different from the first flow channel until the drive unit moves to a position where the drive unit is located when the liquid starts to flow out to the first flow channel.

(11) A filling method, including
discharging a liquid transferred from a liquid transfer pump into a container so as to fill the container through a first flow channel at a predetermined rate, the liquid transfer pump including a flexible tube that allows a liquid to circulate therethrough, a pressing unit that presses the flexible tube from an outer circumferential surface side and feeds the liquid from one end to another end of the flexible tube, and a drive unit that rotationally drives the pressing unit, and then causing the liquid to flow out to a second flow channel different from the first flow channel until the drive unit moves to a position where the drive unit is located when the liquid starts to flow out to the first flow channel.

### REFERENCE SIGNS LIST

1 liquid discharge apparatus
2 liquid tank (liquid storage unit)
3 liquid transfer pump
5 three-way valve (flow channel switching unit)
9 container
11 first tube (first flow channel)
12 second tube (second flow channel)
61 control unit
31 flexible tube
32 eccentric cam (pressing unit)
331 motor (drive unit)
333 encoder (detection unit)

1. A liquid discharge apparatus, comprising:
a liquid transfer pump including
a flexible tube that allows a liquid to circulate therethrough, the liquid being supplied from a liquid storage unit that stores the liquid,
a pressing unit that presses the flexible tube from an outer circumferential surface side and feeds the liquid from one end to another end of the flexible tube, and
a drive unit that rotationally drives the pressing unit;
a first flow channel to which a liquid transferred from the liquid transfer pump flows out;
a second flow channel to which a liquid transferred from the liquid transfer pump flows out, the second flow channel being different from the first flow channel; and
a flow channel switching unit having a flow channel to which a liquid transferred from the liquid transfer pump flows out as the first flow channel or the second flow channel, the flow channel switching unit being controlled to use, as the first flow channel, a flow channel to which a liquid for discharge that is transferred from the liquid transfer pump flows out and use, as the second flow channel, a flow channel to which a liquid transferred from the liquid transfer pump flows out until the drive unit moves to a position after the liquid for discharge is transferred, the position being a position where the drive unit is located when the liquid for discharge starts to flow out.

2. The liquid discharge apparatus according to claim 1, wherein
the drive unit of the liquid transfer pump is driven on a basis of a pulse signal, and
assuming that a number of pulses of the pulse signal for discharge, which is for transferring the liquid for discharge, is defined as m (m>0) and a number of pulses of the pulse signal, which is necessary for the drive unit to rotate once, is defined as n (n>0),
in a case where n<m,
the flow channel switching unit is controlled to use, as the first flow channel, a flow channel to which a liquid transferred from the liquid transfer pump driven on a basis of the pulse signal for discharge flows out and use, as the second flow channel, a flow channel to which a liquid transferred from the liquid transfer pump driven on a basis of the pulse signal with a number of pulses b satisfying m=an+b (a denotes one or more integers, b>0, n>b) flows out after the liquid for discharge is transferred, and
in a case where n>m,
the flow channel switching unit is controlled to use, as the first flow channel, a flow channel to which a liquid transferred from the liquid transfer pump driven on a basis of the pulse signal for discharge flows out and use, as the second flow channel, a flow channel to which a liquid transferred from the liquid transfer pump driven on a basis of a pulse signal with a number of pulses c satisfying c=n−m flows out after the liquid for discharge is transferred.

3. The liquid discharge apparatus according to claim 2, further comprising
a control unit that controls the flow channel switching unit.

4. The liquid discharge apparatus according to claim 3, further comprising
a detection unit that detects a change in position of the drive unit, wherein
the control unit controls the flow channel switching unit on a basis of a detection result of the detection unit.

5. The liquid discharge apparatus according to claim 3, wherein
the control unit further controls an operation of the drive unit, and
the control unit
causes, in a case where n<m, the drive unit to operate on a basis of the pulse signal for discharge and then operate on a basis of the pulse signal with the number of pulses b, and
causes, in a case where n>m, the drive unit to operate on a basis of the pulse signal for discharge and then operate on a basis of the pulse signal with the number of pulses c.

6. The liquid discharge apparatus according to claim 1, wherein
the second flow channel causes a liquid transferred from the liquid transfer pump to flow to the liquid storage unit.

7. The liquid discharge apparatus according to claim 6, wherein
the drive unit is configured to be rotatable in an opposite direction to a direction of rotation during discharging of the liquid.

8. The liquid discharge apparatus according to claim 1, wherein
the first flow channel is constituted by a first tube and the second flow channel is constituted by a second tube, and
the flexible tube, the first tube, and the second tube are configured to be replaceable.

9. The liquid discharge apparatus according to claim 1, wherein
the flow channel switching unit is a pinch valve.

10. A liquid discharge control method, comprising
causing a liquid transferred from a liquid transfer pump to flow out to a first flow channel on a discharge side at a predetermined rate, the liquid transfer pump including a flexible tube that allows a liquid to circulate therethrough, a pressing unit that presses the flexible tube from an outer circumferential surface side and feeds the liquid from one end to another end of the flexible tube, and a drive unit that rotationally drives the pressing unit, and then causing the liquid to flow out to a second flow channel different from the first flow channel until the drive unit moves to a position where the drive unit is located when the liquid starts to flow out to the first flow channel.

11. A filling method, comprising
discharging a liquid transferred from a liquid transfer pump into a container so as to fill the container through a first flow channel at a predetermined rate, the liquid transfer pump including a flexible tube that allows a liquid to circulate therethrough, a pressing unit that presses the flexible tube from an outer circumferential surface side and feeds the liquid from one end to another end of the flexible tube, and a drive unit that rotationally drives the pressing unit, and then causing the liquid to flow out to a second flow channel different from the first flow channel until the drive unit moves to a position where the drive unit is located when the liquid starts to flow out to the first flow channel.
